(19) United States
(12) Patent Application Publication
(10) Pub. No.: US 2024/0350693 A1
(43) Pub. Date: Oct. 24, 2024

(54) SYSTEM AND CARTRIDGE FOR DISPENSING A FRAGRANCE

(71) Applicant: Exuud, Inc., Atlanta, GA (US)

(72) Inventors: Jesse Killon, Alpharetta, GA (US); Abhishek K. Breja, Atlanta, GA (US)

(21) Appl. No.: 18/136,520

(22) Filed: Apr. 19, 2023

(51) Int. Cl. A61L 9/12

(52) U.S. Cl. CPC A61L 9/122; A61L 2209/111; A61L 2209/12; A61L 2209/133

(57) ABSTRACT

A fragrance dispenser, having: a housing defining an air inlet at a bottom of the dispenser and including a cartridge basket at a top of the dispenser; a cartridge removably disposed within the basket, the cartridge including a cartridge housing defining an air inlet at its bottom and an outlet at its top, and including a tray stack of circular trays configured the same as each other, each of the circular trays having at least one bin configured to receive a fragrance member; a tray drive shaft within the cartridge configured to sequentially rotate each of the trays about a tray stack center; a drive unit coupled to the tray drive shaft; and an air intake unit that draws air into the air inlet of the dispenser, into the air inlet of the cartridge, through the cartridge, and out of the air outlet of the cartridge, while the trays rotate.

### BACKGROUND

The embodiments herein relate to fragrance dispensers and more specifically to a system and cartridge for dispensing a fragrance.

A wide variety of devices may be used for dispensing both liquid and solid fragrances to the environment of rooms, automobiles, and the like. Long-lasting fragrance dispensers may consist of a housing within which a reservoir of fragrance is held, which gradually evaporates fragrance into the surrounding air. The reservoir is often defined within a cartridge that can be removed from the housing when exhausted and replaced. It is desirable to increase the longevity of the fragrance availability within the cartridge.

Accordingly, while existing fragrance dispensers were suitable for their intended purposes the need for improvement remains, particularly in providing a fragrance dispenser having one or more of the features described herein.

### BRIEF SUMMARY

Disclosed is a system for dispensing a fragrance, including: a housing extending from a bottom of the dispenser to a top of the dispenser, defining an air inlet at the bottom and including a cartridge basket at the top; a cartridge removably disposed within the cartridge basket, the cartridge including a cartridge housing extending from a bottom to a top and defining an air inlet at the bottom and an outlet at the top, the cartridge including a tray stack of circular trays configured the same as each other, each of the circular trays having at least one bin configured to receive a fragrance member; a tray drive shaft, within the cartridge, configured to sequentially rotate each of the trays about a tray stack center; a drive unit with the dispenser, operationally coupled to the tray drive shaft; and an air intake unit configured to draw air into the air inlet of the dispenser, into the air inlet of the cartridge, through the cartridge, and out of the air outlet of the cartridge, while the drive unit rotates the trays.

In addition to one or more of the features described herein, or as an alternative, further embodiments of the dispenser may include: the air inlet in the cartridge housing includes a center opening and an annular- or ring-section shaped opening radially offset and spaced apart from the center opening with a first circumferential span; and each of the trays includes a center opening and an annular- or ring-section shaped first segment with first airflow passages, the first segment forming at least one bin, the bin having a second circumferential span.

In addition to one or more of the features described herein, or as an alternative, further embodiments of the dispenser may include: the tray drive shaft, is configured to sequentially rotate each of the trays about the tray stack center by the circumferential span of one bin such that, in an initial rotational state and a final rotational state, the bins of each of the trays are circumferentially aligned with each other and with the offset opening of the cartridge housing.

In addition to one or more of the features described herein, or as an alternative, further embodiments of the dispenser may include: each of the trays extends from a tray bottom to a tray top by a tray height; and the walls of the bins in the first segment each extend from a tray bottom to a tray top; and the floor of the bins has openings to allow airflow but that will not allow fragrance members to pass through and has a height less than the tray height, the difference between the tray height and the floor height defining a bin depth.

In addition to one or more of the features described herein, or as an alternative, further embodiments of the dispenser may include: bins in a second segment of a tray are configured to prevent the placement of the fragrance members therein.

In addition to one or more of the features described herein, or as an alternative, further embodiments of the dispenser may include: each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and the tray drive shaft extends axially through the tray stack, within the shaft opening, and is radially smaller than the inner diameter wall, so that a rotation axis of the tray drive shaft is offset from the tray stack center, the tray drive shaft having teeth that are axially spaced apart from each other for engaging the inward facing gear teeth of each of the respective ones of the trays.

In addition to one or more of the features described herein, or as an alternative, further embodiments of the dispenser may include: each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and the inward facing gear teeth of each of the trays engages an independently rotating idler gear, the axially spaced stack of idler gears extending axially through the tray stack, within the shaft opening, the idler gears being radially smaller than the inner diameter wall, so that a rotation axis of the idler gears is offset from the tray stack center; and the tray driveshaft having an axial stack of interrupted gears engaging each idler gear, the tray drive shaft extends axially through the tray stack, within the shaft opening, and is radially smaller than the inner diameter wall, so that a rotation axis of the tray drive shaft is offset from the tray stack center.

In addition to one or more of the features described herein, or as an alternative, further embodiments of the dispenser may include: the stack of trays has a first number of the trays, and the tray drive shaft has a second number of the axially spaced interrupted gears that is the same as the first number, and the number of idler gears, if present, is the same as the first number.

In addition to one or more of the features described herein, or as an alternative, further embodiments of the dispenser may include: each of the axially spaced interrupted gears on the driveshaft has a set of gear teeth of at least one tooth, spanning a circumferential segment less than 360 degrees for engaging the respective idler gear teeth, if present, or engaging the respective tray teeth for rotating the idler or tray when the teeth are engaged.

In addition to one or more of the features described herein, or as an alternative, further embodiments of the dispenser may include: each of the axially spaced interrupted gears on the driveshaft has an extended ring or circular plate spanning a circumferential segment less than 360 degrees for engaging the teeth or semicircular grooves on the idler gears, if present, or engaging the teeth or semi-circular grooves in the tray to prevent the idler or tray from rotating while the ring or plate is engaged.

In addition to one or more of the features described herein, or as an alternative, further embodiments of the dispenser may include: each set of teeth on the tray drive shaft interrupted gears are angularly offset from the teeth that are adjacent to it on the tray drive shaft such that the teeth of each interrupted gear engages the teeth of the respective idler gears or trays in sequence.

In addition to one or more of the features described herein, or as an alternative, further embodiments of the dispenser may include: each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and the inward facing gear teeth on the trays have a blocked segment with a first circumferential span and a cut away segment with a second circumferential span, the first segment being placed to prevent the driveshaft or idler gear teeth from engaging the tray before the degree of initial rotation of the tray within the cartridge, the second segment being placed so that at the final degree of rotation, i.e. when the tray has rotated such that the last unused bin is positioned above the offset opening in the cartridge, the idler gear or driveshaft gear is positioned within the cutout and the teeth of the tray are disengaged from the teeth of the idler or driveshaft.

In addition to one or more of the features described herein, or as an alternative, further embodiments of the dispenser may include: the dispenser includes an energy storage device and a reduction gear powered by the energy storage device, wherein the reduction gear includes an output gear; and the tray drive shaft includes a bottom end that is formed with spline teeth and configured for engagement with the output spline of the reduction gear.

In addition to one or more of the features described herein, or as an alternative, further embodiments of the dispenser may include: a tray cap of the cartridge is disposed above the top tray, the tray cap having a same size as the trays, the tray cap defines an offset opening that is aligned with the offset opening of the cartridge housing; and cartridge louvers are pivotally coupled to the baseplate at the offset opening of the tray cap and configured to pivot between a closed state in which the offset opening of the tray cap is blocked and an open state in which the offset opening of the tray cap is exposed, wherein the cartridge louvers are air biased to transition from the closed state to the open state, and are otherwise in the closed state.

In addition to one or more of the features described herein, or as an alternative, further embodiments of the dispenser may include: a top plate of the cartridge is disposed above the tray cap, the top plate having a center opening the same size as the center opening in the top cap, and the top cap having a center opening the same size as the center opening in the tray stack and an offset opening that is aligned with the offset opening of the cartridge housing, the combination of tray cap and top plate thus configured to guide air flowing through the offset opening and through the first or second segments of the tray stack to combine with air flowing through the center opening of the tray stack before flowing out of the cartridge.

In addition to one or more of the features described herein, or as an alternative, further embodiments of the dispenser may include: the cartridge includes memory that stores flash memory data indicative of one or more of a type of fragrance member stored within the cartridge, fragrance member usage history, target speeds of the air movers, and/or a target speed of the tray drive shaft; and the dispenser includes a processor that is configured to communicate with the memory on the cartridge and control a speed of the air movers, and/or the output gear responsive to the flash memory data.

In addition to one or more of the features described herein, or as an alternative, further embodiments of the dispenser may include: the dispenser includes a fan configured to draw air into the inlet, and a motion sensor, and the processor in the dispenser is configured to activate the fan upon the motion sensor detecting motion.

In addition to one or more of the features described herein, or as an alternative, further embodiments of the dispenser may include: the dispenser includes a fan configured to draw air into the inlet, and a motion sensor, and the processor in the dispenser is configured to predictively activate the fan based upon the timing history of signals from the motion sensor detecting motion and/or a preset schedule from a connected software application and/or processor firmware. In one or more embodiments, the predictive activation of the fan is based on machine learning.

In addition to one or more of the features described herein, or as an alternative, further embodiments of the dispenser may include: the dispenser includes an LED array at the top of the housing and an LED array controller that is operationally connected to the processor in the housing and configured to illuminate upon settings from a software application, settings from capacitively sensed gestures on the device, and/or the motion sensor detecting motion.

In addition to one or more of the features described herein, or as an alternative, further embodiments of the dispenser may include: the dispenser includes an LED array configured to illuminate the top of the cartridge.

In addition to one or more of the features described herein, or as an alternative, further embodiments of the dispenser may include: the dispenser includes a ring-shaped lip under which a capacitive touch sensing circuit board is used to detect gestures by the user on the ring to adjust the operational settings for the dispenser.

In addition to one or more of the features described herein, or as an alternative, further embodiments of the dispenser may include: the dispenser includes a baseplate disposed below the cartridge basket, the baseplate extends between an outer diameter edge and an inner diameter edge that are radially spaced apart from each other, the inner diameter edge defines a fan opening; and a center fan is disposed with the fan opening, operationally connected to the processor in the dispenser, and configured to direct air through the shaft opening of the tray stack.

In addition to one or more of the features described herein, or as an alternative, further embodiments of the dispenser may include: the baseplate of the dispenser defines an offset opening that is aligned with and with the offset opening of the cartridge housing, and a second fan is disposed below the offset opening, operationally connected to the processor in the dispenser, and configured to direct air through the offset opening.

In addition to one or more of the features described herein, or as an alternative, further embodiments of the dispenser may include: housing louvers are pivotally coupled to the cartridge basket at the offset opening in the cartridge basket and configured to pivot between a closed state in which the offset opening is blocked and an open state in which the offset opening is exposed, wherein the housing louvers are air biased by the second fan to transition from the closed state to the open state, and are otherwise in the closed state.

Also disclosed is a cartridge for dispensing a fragrance, including: a cartridge housing extending from a bottom to a top, and defining an air inlet at the bottom and an outlet at the top; a tray stack of circular trays configured the same as each other, each of the circular trays having at least one bin configured to receive a fragrance member; a tray drive shaft, within the cartridge, configured to sequentially rotate each of the trays about a tray stack center, wherein the tray drive shaft is configured for being driven by an external drive unit.

In addition to one or more of the features described herein, or as an alternative, further embodiments of the cartridge may include: the air inlet of the cartridge housing includes a center opening and a radially offset opening with a first circumferential span that is spaced apart from the center opening; and each of the trays extends between an outer diameter and an inner diameter that are radially spaced apart from each other; and each of the trays includes a first segment having a first circumferential span and first airflow passages, and a second segment having a second circumferential span and second airflow passages, the first segment forming at least one bin.

In addition to one or more of the features described herein, or as an alternative, further embodiments of the cartridge may include: the tray drive shaft is configured to sequentially rotate each of the trays about the tray stack center by the circumferential span of one bin such that, in an initial rotational state and a final rotational state, the bins of each of the trays are circumferentially aligned with each other and the offset opening the cartridge housing.

In addition to one or more of the features described herein, or as an alternative, further embodiments of the cartridge may include: each of the trays extends from a tray bottom to a tray top by a tray height; and the walls forming the bins of the first segment of each of the trays extend from a tray bottom to a tray top, and the floor of the bins has openings to allow airflow but that will not allow fragrance members to pass through and has a height less than the tray height, the difference between the tray height and the floor height defining a bin depth.

In addition to one or more of the features described herein, or as an alternative, further embodiments of the cartridge may include: bins in a second segment are configured to prevent the placement of the fragrance members therein.

In addition to one or more of the features described herein, or as an alternative, further embodiments of the cartridge may include: each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and the tray drive shaft extends axially through the tray stack, within the shaft opening, and is radially smaller than the inner diameter wall, so that a rotation axis of the tray drive shaft is offset from the tray stack center, the tray drive shaft having teeth that are axially spaced apart from each other for engaging the inward facing gear teeth of each of the respective ones of the trays.

In addition to one or more of the features described herein, or as an alternative, further embodiments of the cartridge may include: each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and the inward facing gear teeth of each of the trays engages an independently rotating idler gear, the axially spaced stack of idler gears extending axially through the tray stack, within the shaft opening, the idler gears being radially smaller than the inner diameter wall, so that a rotation axis of the idler gears is offset from the tray stack center; and the tray driveshaft having an axial stack of interrupted gears engaging each idler gear, the tray drive shaft extends axially through the tray stack, within the shaft opening, and is radially smaller than the inner diameter wall, so that a rotation axis of the tray drive shaft is offset from the tray stack center.

In addition to one or more of the features described herein, or as an alternative, further embodiments of the cartridge may include: the stack of trays has a first number of the trays, and the tray drive shaft has a second number of the axially spaced interrupted gears that is the same as the first number, and the number of idler gears, if present, is the same as the first number.

In addition to one or more of the features described herein, or as an alternative, further embodiments of the cartridge may include: each of the axially spaced interrupted gears on the driveshaft has a set of gear teeth, at least one tooth, spanning a circumferential segment less than 360 degrees for engaging the respective idler gear teeth, if present, or engaging the respective tray teeth for rotating the idler or tray when the teeth are engaged.

In addition to one or more of the features described herein, or as an alternative, further embodiments of the cartridge may include: each of the axially spaced interrupted gears on the driveshaft has an extended ring or circular plate spanning a circumferential segment less than 360 degrees for engaging the teeth or semicircular grooves on the idler gears, if present, or engaging the teeth or semi-circular grooves in the tray to prevent the idler or tray from rotating while the ring or plate is engaged.

In addition to one or more of the features described herein, or as an alternative, further embodiments of the cartridge may include: each set of teeth on the tray drive shaft interrupted gears are angularly offset from the teeth that are adjacent to it on the tray drive shaft such that the teeth of each interrupted gear engage the teeth of the respective idler gears or trays in sequence.

In addition to one or more of the features described herein, or as an alternative, further embodiments of the cartridge may include: a tray cap of the cartridge is disposed above the top tray, the tray cap having a same size as the trays, the tray cap defines an offset opening that is aligned with the offset opening of the cartridge housing; and cartridge louvers are pivotally coupled to the baseplate at the offset opening of the tray cap and configured to pivot between a closed state in which the offset opening of the tray cap is blocked and an open state in which the offset opening of the tray cap is exposed, wherein the cartridge louvers are air biased to transition from the closed state to the open state, and are otherwise in the closed state.

In addition to one or more of the features described herein, or as an alternative, further embodiments of the cartridge may include: a top plate of the cartridge is disposed above the tray cap, the top plate having a center opening the same size as the center opening in the top cap, and the top cap having a center opening the same size as the center opening in the tray stack and an offset opening that is aligned with the offset opening of the cartridge housing, the combination of tray cap and top plate thus configured to guide air flowing through the offset opening and through the first or second segments of the tray stack to combine with air flowing through the center opening of the tray stack before flowing out of the cartridge.

In addition to one or more of the features described herein, or as an alternative, further embodiments of the cartridge may include: the tray drive shaft includes a bottom end that is formed with spline teeth and configured for engagement with a mating spline.

In addition to one or more of the features described herein, or as an alternative, further embodiments of the cartridge may include: each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and the inward facing gear teeth on the trays have a blocked segment with a first circumferential span and a cut away segment with a second circumferential span, the first segment being placed to prevent the driveshaft or idler gear teeth from engaging the tray before the degree of initial rotation of the tray within the cartridge, the second segment being placed so that at the final degree of rotation; i.e. when the tray has rotated such that the last unused bin is positioned above the offset opening in the cartridge, the idler gear or driveshaft gear is positioned within the cutout and the teeth of the tray are disengaged from the teeth of the idler or driveshaft.

In addition to one or more of the features described herein, or as an alternative, further embodiments of the cartridge may include: the cartridge includes memory that stores flash memory data indicative of one or more of a type of fragrance member stored within the cartridge, fragrance member usage history, target speeds of the air movers, and/or a target speed of the tray drive shaft.

These and other advantages and features will become more apparent from the following description taken in conjunction with the drawings.

### BRIEF DESCRIPTION OF THE DRAWINGS

The present disclosure is illustrated by way of example and not limited in the accompanying figures in which like reference numerals indicate similar elements.

FIG. 1 is a perspective view of a fragrance dispenser according to an embodiment;

FIG. 2 is another perspective view of the fragrance dispenser showing a fragrance cartridge therein;

FIG. 3 is a perspective view of the fragrance dispenser with an outer housing not shown;

FIG. 4 is a cross sectional view of the fragrance dispenser;

FIG. 5 is a perspective view of the fragrance dispenser with an outer housing not shown with local cross sections to show detail;

FIG. 6 is a top perspective view of the fragrance dispenser showing a cartridge receiving basket and louvers;

FIG. 7 is a bottom perspective view of the cartridge;

FIG. 8 and FIG. 9 are perspective views of the internal mounting plate of the fragrance dispenser, with certain aspects not shown, and showing internal fans, motor, and reduction gear train;

FIG. 10 is two bottom perspective views of the fragrance dispenser cartridge receiving basket and upper printed circuit board (PCB), with certain aspects not shown, and with the light guide and LED array shown;

FIG. 11 and FIG. 12 are perspective and cross-sectional views of the fragrance cartridge according to an embodiment;

FIG. 13 and FIG. 14 are a perspective and top view of the tray stack, driveshaft, and idler gears of the fragrance cartridge;

FIG. 15 is an enlarged view of a portion of FIG. 13;

FIG. 16 is an enlarged view of a portion of FIG. 14;

FIG. 17 is a top perspective view of the fragrance cartridge, with certain aspects not shown, and with the inner tray stack cap and louvers shown;

### DETAILED DESCRIPTION

A detailed description of one or more embodiments of the disclosed apparatus and method are presented herein by way of exemplification and not limitation with reference to the Figures.

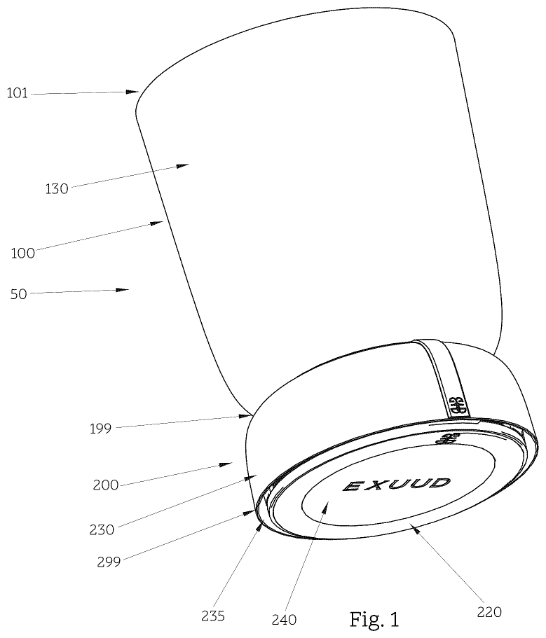

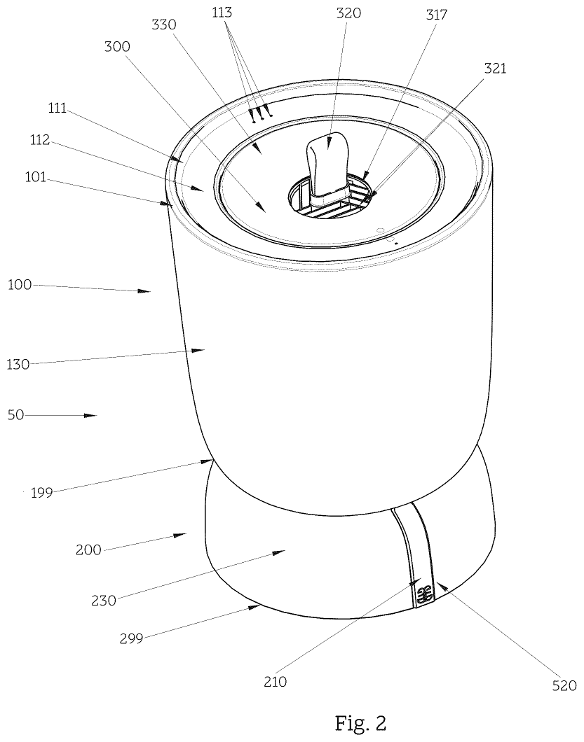

Turning to FIGS. 1 and 2, a system 50 for dispensing a fragrance is shown (otherwise referred to as a dispenser). The dispenser 50 has a bottom (or lower) portion 200 and a top (or upper) portion 100. The bottom portion 200 of the dispenser 50 extends from a bottom 299 of the dispenser 50 to an intermediate location or junction 199 between the portions 200, 100. The top portion 100 extends from the intermediate location 199 to a top 101 of the dispenser 50 and is axially longer than the bottom portion 200. A removable cartridge 300 is installed through the top 101 of the dispenser 50, into the top portion 100.

The upper portion 100 of the dispenser 50 includes an outer housing 130, alternatively referred to as a housing or a shell, extending from the intermediate junction 199 to the top 101 of the dispenser 50. The lower portion 200 of the dispenser 100 includes and outer shell 230, alternatively referred to as a housing or shell, forming the outer surface of the lower portion extending from the bottom 299 to the intermediate junction 199 of the device 50. Both shells 130 & 230 may be substantially cylindrical or frustoconical with rounded ends at the junction 199.

At the top 101 of the dispenser 50, a ring-shaped lip 112 surrounds the cartridge 300. The ring-shaped lip, 112, may conceal a touch sensitive component that allows the user to control the dispenser 50. For example, it may be a capacitive device. It may have indicator lights 113 to display the current operational settings.

The lower portion 200 of the dispenser 50 includes a lower frame 240 which forms the foundation that the dispenser sits on. A gap 235 between the lower shell 230 and the lower frame 240 forms an air inlet for the dispenser 50. A window 210 is formed within the bottom portion 200, behind which is a motion sensor 520 for controlling the dispenser 50. That is, optionally, when motion is sensed, the dispenser 50 may change from standby to active modes and/or dispenses fragrance at a greater or lesser rate based on the motion sensor reading.

The cartridge 300 includes a top opening 317 that forms an air outlet or vent of the dispenser 50. The cartridge 300 includes a lifting strap 320 for insertion and removal of the cartridge 300 within the dispenser 50. As described in more detail below, the top of the cartridge 330 is configured to illuminate when the device 50 is turned on. The illumination can be controlled by the user via the touch-sensitive ring 112, via a mobile software app that connects to the device's controller 510 wirelessly, or by the motion sensor 520, described in more detail below.

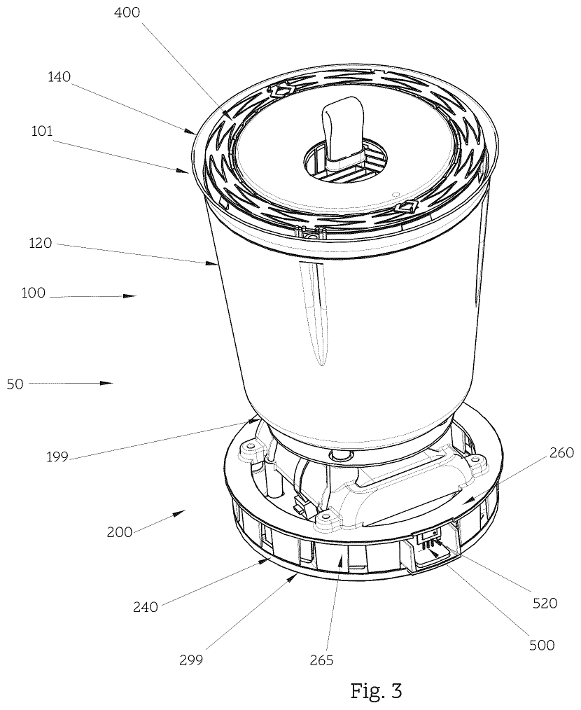

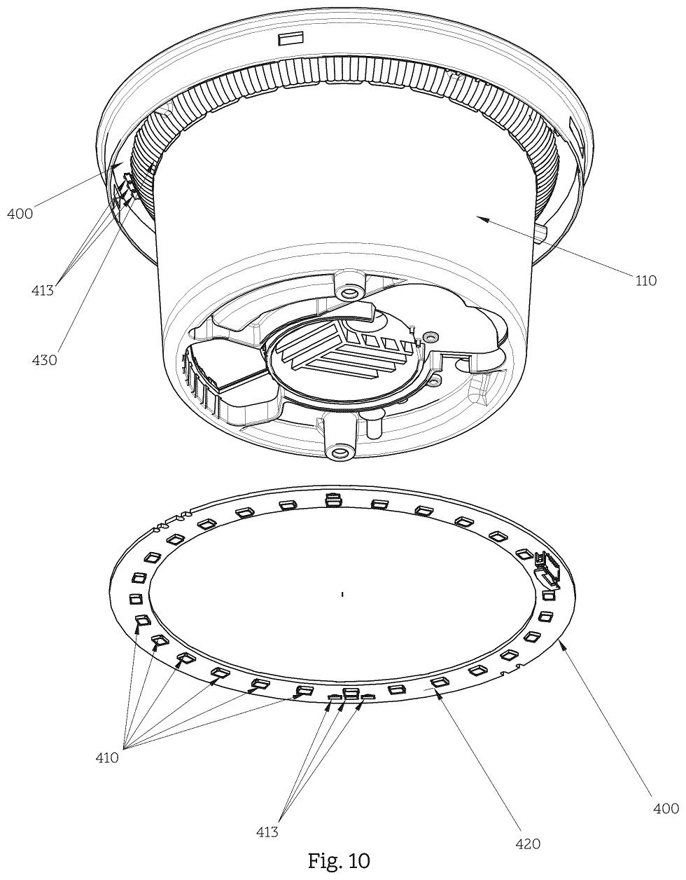

Turning to FIGS. 3, 4, and 5, an inner housing 120, is located within the top portion 100 and extends from the junction 199 toward the top 101 of the dispenser 50 and supports an upper circuit board 400 and a cartridge receiving basket 110. The cartridge receiving basket 110 is a generally cylindrical structure extending from a base 119 to a top 111; the top comprising the touch-sensitive ring-shaped lip 112. The upper circuit board 400 is held under the ring-shaped lip 112 of the cartridge receiving basket 110 to provide touch-sensitivity. The upper circuit board 400 includes an LED array 410, or LED ring, and LED array controller 420 (FIG. 10, discussed below) that may be controlled by the motion sensor 520, the touch-sensitive circuit board 400 of the dispenser 50, or a wirelessly connected software application. The bottom of the inner housing 120 defines a circular airway opening 125 that receives air from the dispenser inlet 235. A flexible top gasket 140 near the top 101 of the dispenser 50 extends between the inner housing 120 and the upper shell 130. The gasket 140 provides advantages in concealing dimensional variations between the cartridge receiving basket 110 and the outer shell 130 to provide a more visually appealing appearance. A baseplate 600 is attached to the bottom of the cartridge receiving basket 110. To the baseplate is attached an off-center air mover 660, which may be a centrifugal blower for example, a center air mover 650, which may be an axial fan for example, a motor 629 that drives a gear train 630 with an output driveshaft 640, and an intermediate printed circuit board (PCB) 680.

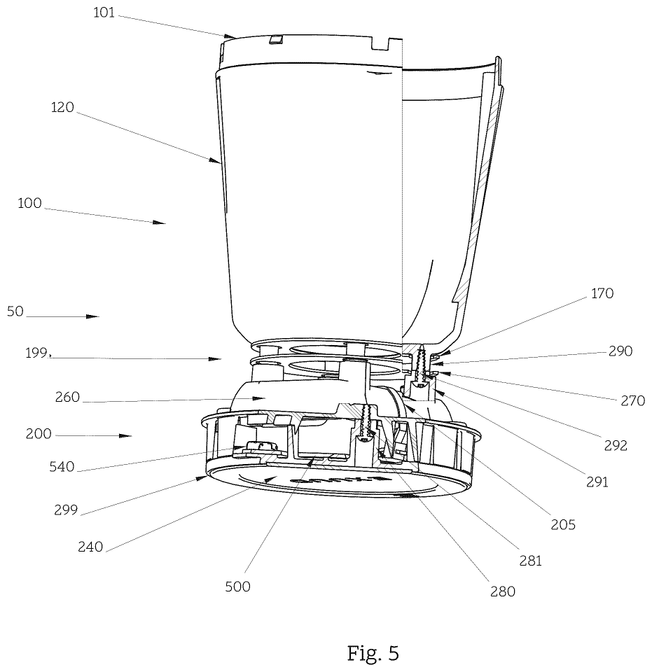

The bottom portion 200 of the dispenser 50 includes a flat, ring-shaped elastomer foot 220, which supports the device. The base foot may be attached to a lower base frame 240. The lower base frame 240 may be fastened, e.g., by screws 281 (FIG. 5) through molded-in standoffs 280 extending axially to an upper base frame 260. The upper base frame includes intake vanes 265 that form a labyrinth air intake configuration to prevent direct viewing of the internal device.

The upper base frame 260 may be attached to the inner housing 120, e.g. by screws 292 (FIG. 5) through axial standoffs 291 in the upper base frame 260. The screws 292 may attach to corresponding axial standoffs 290 in the inner housing 120. Gasket rings 170, 270 may surround the axial posts 290, 291 and compressively locate and support the upper outer housing 130.

A lower circuit board 500 with a system controller or processor 510 is attached above the base lower frame 240. An energy storage device 530 (e.g. a battery) is within the bottom portion 200 as well as a charge port 540 for charging the energy storage device 530. The energy storage device 530, upper circuit board 400, intermediate circuit board 680, center air mover 650, off-center air mover 660, and motor 629 are operatively coupled to the lower circuit board 500. In an embodiment, the energy storage device 530 includes a cover having batteries therein. A strap 205 keeps the energy storage device 530 attached to an upper housing support 260 that includes a pocket for the energy storage device 530.

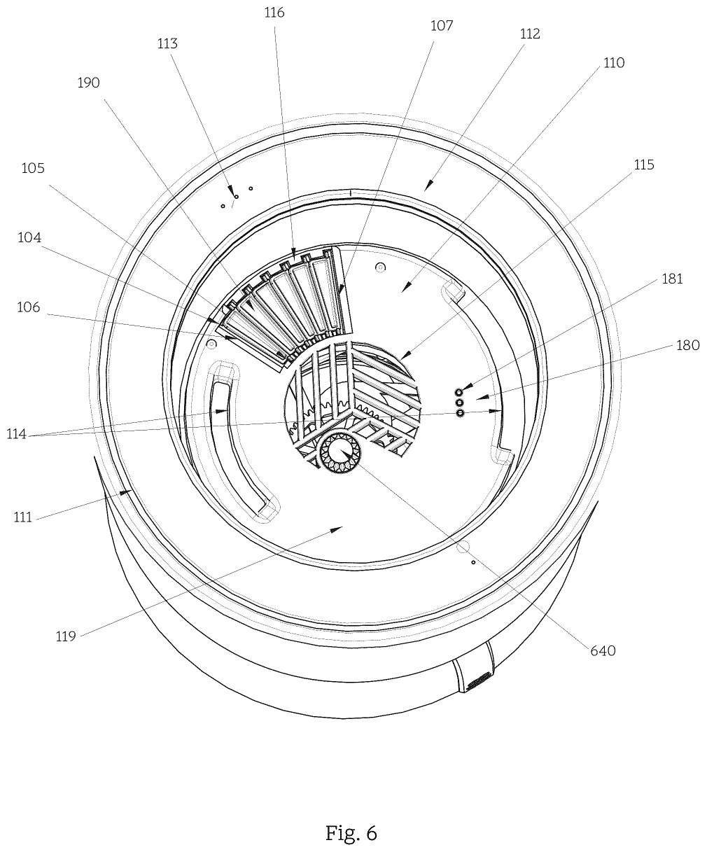

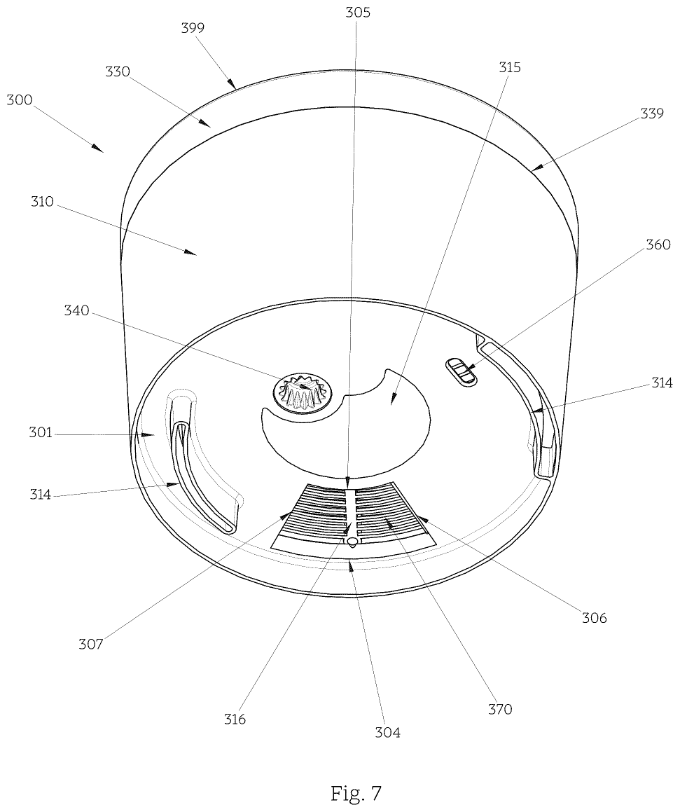

Turning to FIGS. 6 & 7, an inside of the cartridge basket 110 (FIG. 6) is shown from above and a cartridge 300 is shown from below (FIG. 7). The cartridge 300 is removably disposed within the cartridge basket 110 of the dispenser 50. The cartridge basket 110 extends from a base 119 to a top 111 and is keyed with slot features 114 in its base 119. The cartridge 300 extends from its base 301 to its top 399. Projections 314 formed into the base 301 of the cartridge 300 match the slot features 114 in the cartridge basket 110 so that the cartridge 300 aligns with a predetermined orientation when it is removably disposed within the dispenser 50. The cartridge lower housing 310 may include molded in ferrous inserts and the cartridge basket 110 may include magnets for attracting the ferrous inserts so that the cartridge 300 is attractively held in place when it is removably disposed in its predetermined orientation in the dispenser 50.

The cartridge basket 110 has a base 119 with a center opening 115 and an offset opening 116, alternatively referred to as off-center opening. The offset opening 116 is shaped like a ring sector or annulus sector. The offset opening 116 is formed by an outer radius 104; an inner radius 105 near the center opening 115, and circumferential boundaries 106 & 107. In an embodiment, the angle circumscribed between circumferential boundaries 106 & 107 of the offset opening 116, is between 10 and 180 degrees. In another embodiment, the angle is between, between 10 and 90 degrees. In still another embodiment, the angle is between 30 and 60 degrees. The cartridge 300 includes a cartridge lower housing 330 extending from a bottom 301 to an intermediate junction 339 and has two air inlets on the bottom 301: a center opening 315 and an offset opening 316, alternatively referred to as off-center opening. Openings 115, 116 in the cartridge basket 110 align with openings 315, 316 in the base of the cartridge 300 when the cartridge is removably disposed in the predetermined orientation in the device 50. The cartridge housing 300 includes a top cover 330 (FIG. 2) extending from an intermediate junction 339 to a top 399 and has a center air outlet 317 (FIG. 2) at the top 399. The cartridge 300 also includes the lifting strap 320. Vanes 321 (FIG. 2) span the center opening 317 in the top cover 330 support the lifting strap 320.

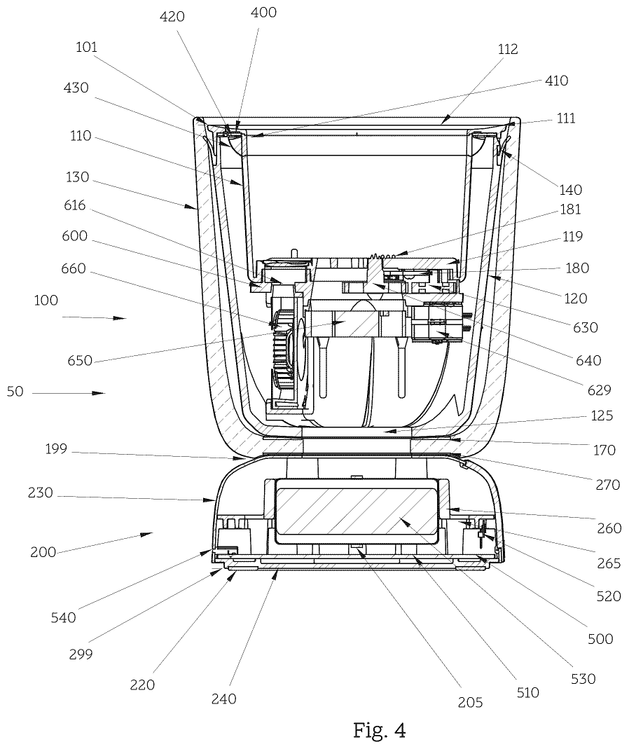

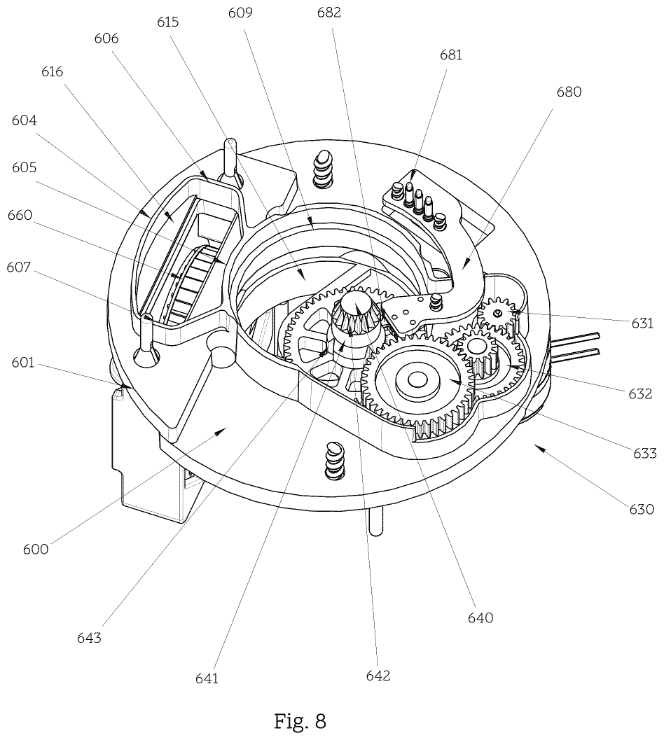

Housing louvers 190 are pivotally coupled to the base 119 at the offset opening 116 in the cartridge basket 110, so that the housing louvers 190 are between the baseplate 600 (FIGS. 4, 8) and the cartridge 300. The housing louvers 190 are configured to pivot between a closed state in which the offset opening 116 is blocked and an open state in which the offset opening 116 is open. The housing louvers 190 are air biased by the operation of off-center air-mover 660 (FIG. 8) to transition from the closed state to the open state. The housing louvers 190 are otherwise in the closed state. In an embodiment, the housing louvers 190 move to the closed state under the influence of gravity. It is to be appreciated that the opening 316 in the cartridge base 301 is positioned to allow for motion of the housing louvers 190.

Spring-loaded electrical leads 681 (also FIG. 4, FIG. 8) extend from an intermediate PCB 680 (FIGS. 4, 6) through cartridge basket 110 and through a slot in the base 301 of the cartridge lower housing 310 for receiving data from a solid-state memory device 360 (FIG. 7) within the cartridge 300, such as a flash memory for example. The flash memory 360 stores data which may be indicative of one or more of a type of fragrance member stored within the cartridge, target speeds of the dispenser fans 660 & 650, target time interval for the tray drive shaft 340, and the history of use of the cartridge. The processor 510 in the dispenser 50 is configured to communicate with the memory 360 of the cartridge 300. The processor uses data from the memory 360 along with user-selected operating settings and data from the motion sensor 520 to control timing of the output gear driveshaft 640 and speed of the dispenser fans 660 & 650. In an embodiment, the processor uses machine learning to predict when the fans 660, 650 may be activated. In an embodiment, the machine learning may be trained at least in part on the timing history of signals from motion sensor 520.

An output gear drive shaft 640 with an external tapered spline extends through the base 119 of the cartridge basket 110. A driven shaft 340 with an internal tapered spline in the cartridge 300 mates with the drive shaft spline 640 when the cartridge 300 is removably disposed in the predetermined orientation in the device 50.

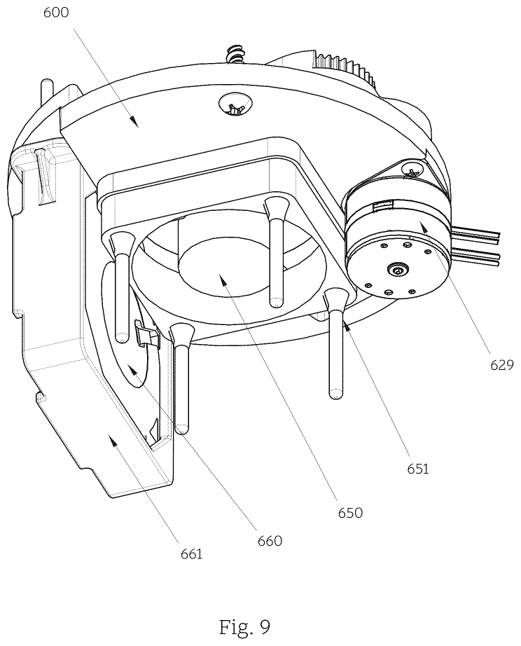

Turning to FIG. 8 and FIG. 9, a baseplate 600 extends between an outer diameter edge 601 and an inner diameter edge 609 that are radially spaced apart from each other. The inner diameter edge 609 defines the center opening 615. The center opening 615 is a frustoconical section that circumscribes the outlet of a center air mover 650, which in an embodiment may be an axial fan, on one end and the center opening 115 of the cartridge basket 110 on the other end. An off-center opening 616 is defined by edges at an outer radius 604, an inner radius 605, and two sides 606 and 607. The off-center opening 616 is shaped to circumscribe the outlet of an off-center air mover 660, which may be a centrifugal blower, and the off-center opening 116 in the cartridge basket 110.

A reduction gear train 630, driven by the energy storage device 530 via a motor 629, is supported on the baseplate 600. The gear train may have an input gear 631 directly driven by the motor 629, intermediate gears 632, 633, and an output gear driveshaft 640 which has an extended axial shaft 641 with an external tapered spline 642 on the end. The extended shaft 641 may also have timing marks 643 on its outer diameter that engage a microswitch 682 on an intermediate PCB 680 to provide feedback to the microcontroller 510 driving the motor 629 to signal when target rotation of the driveshaft 640 has been reached.

An off-center centrifugal fan 660 is supported below the baseplate 600 below the off-center opening 616 by a vibration isolating mount 661. A center axial fan 650 is supported in the center opening 615 of the baseplate 600 by a vibration-isolating mount 651. The center fan 650 and off-center fan 660 may be alternatively referred to as first and second fans or primary and secondary fans. The fans 650, 660 form an air intake unit that is configured to draw air into the air inlet 235 of the dispenser 50 and through the cartridge 300, and out of the air outlet 317 of the dispenser 50. The baseplate 600 and the cartridge basket 110 may have mating axial walls around the center openings 115, 315 and off-center openings 116, 316 that create a ducted airflow pathway between the center and off-center air movers 650, 660 and the center and off-center cartridge openings 315, 316, respectively. The off-center fan may be used to control the diffusion rate of the fragrance from the cartridge. The center fan 650 may be used to control the diffusion rate of the fragrance-loaded air into the surroundings. The fans 650, 660 are controlled to operate based on schedules selected by the user via wirelessly connected software application, schedules stored in the cartridge memory chip 360, when the motion sensor 520 detects motion, or combinations thereof. The gear reduction 630 mechanism operates periodically to actuate the driveshaft 340 of the cartridge 300 based on usage rates of the fans 650, 660, parameters stored in the cartridge memory 360, and the wirelessly connected software application.

Turning to FIG. 10, a touch-sensitive ring PCB 400 located under the ring-shaped lip 112 of the cartridge basket 110 is shown. A transparent lens, or light guide, 430 which may have reflective surfaces, is mounted below the ring PCB 400 (see also FIG. 4). An LED array 410 and LED array controller 420 are mounted on the bottom of the ring PCB 400. The LED array 410 emits light into the light guide 430 which then turns the light to a largely radial direction. This light passes through a transparent window in the cartridge basket 110 and is directed into the upper housing 330 of the cartridge 300 to provide illumination on the top of the cartridge 300. Indicator LED emitters 413 are oriented to emit light up through the ring-shaped lip 112 of the cartridge basket to give visual information about the device settings to the user.

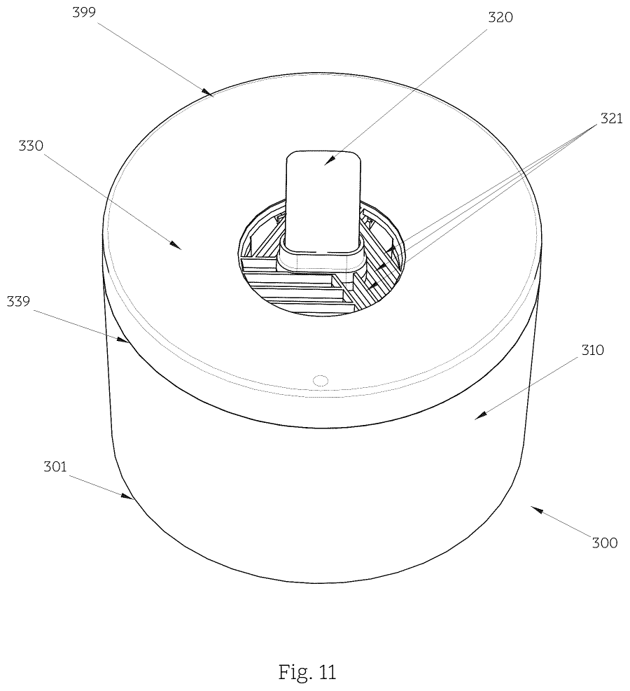

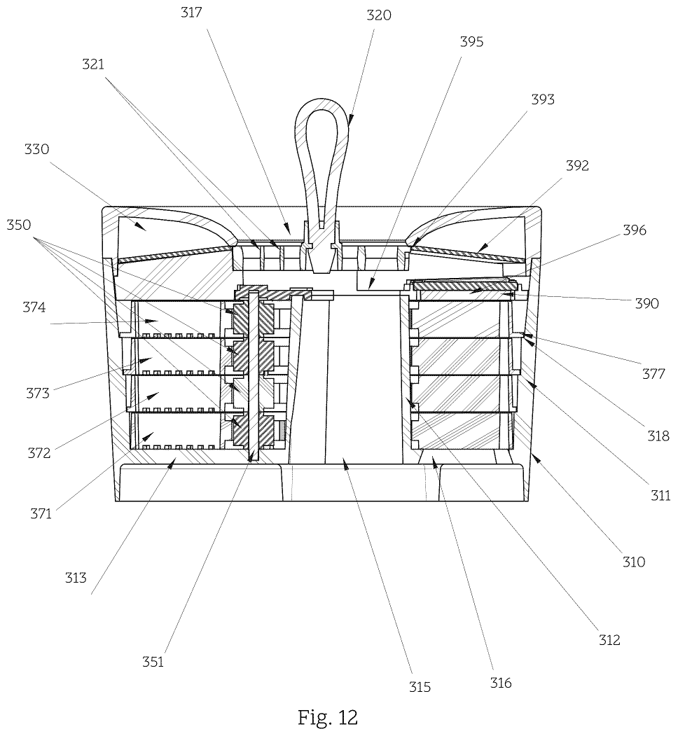

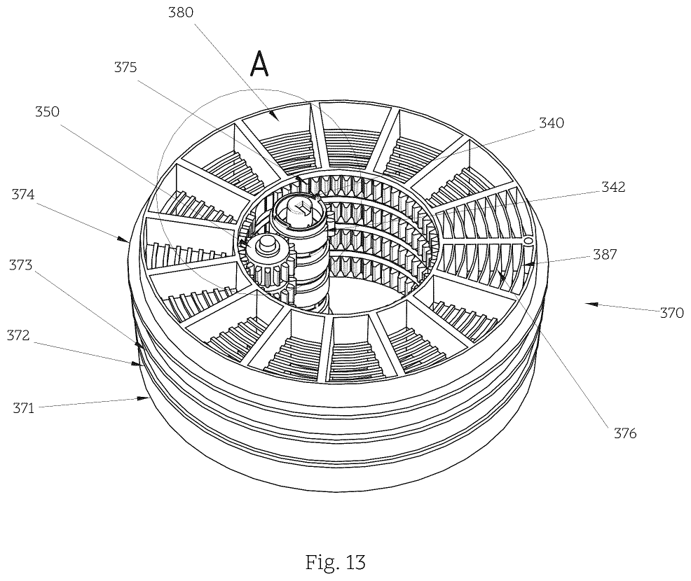

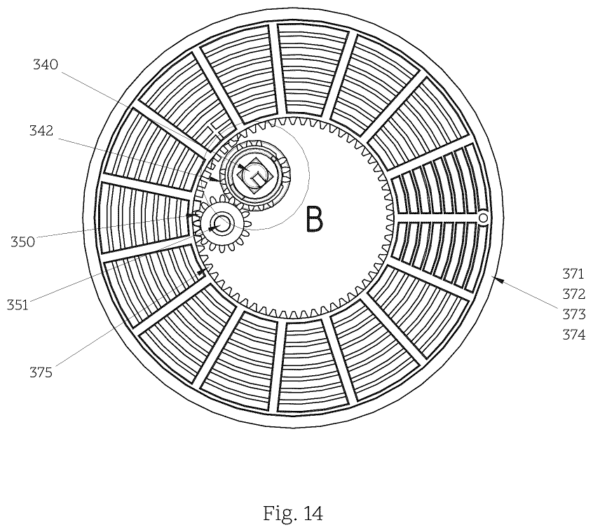

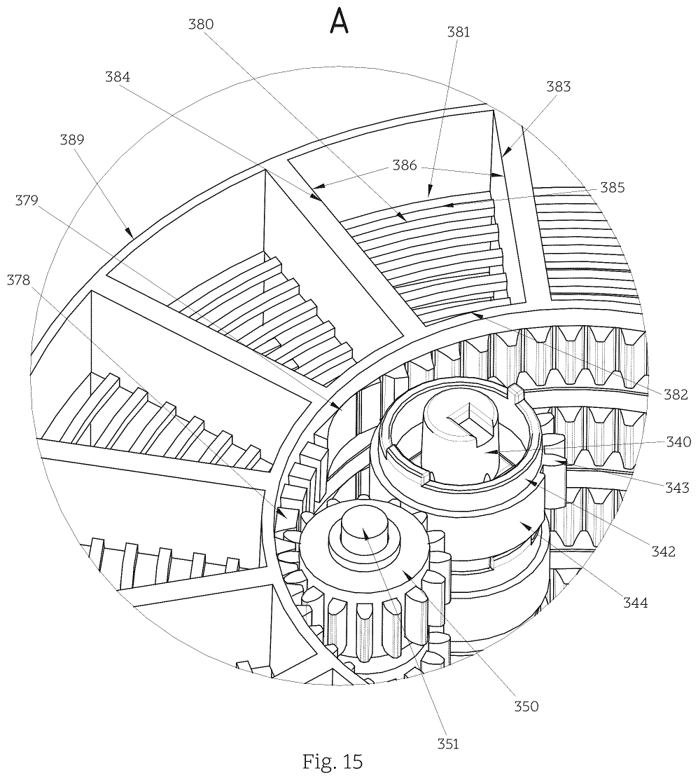

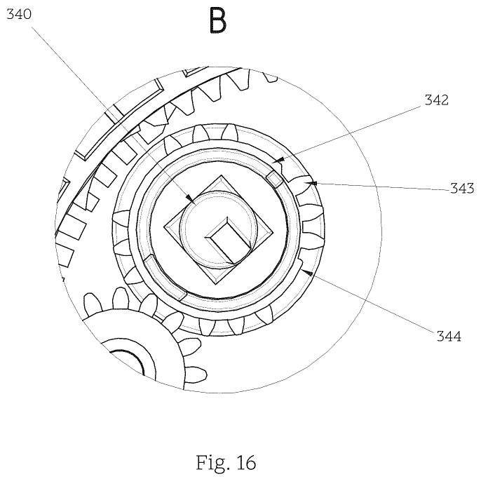

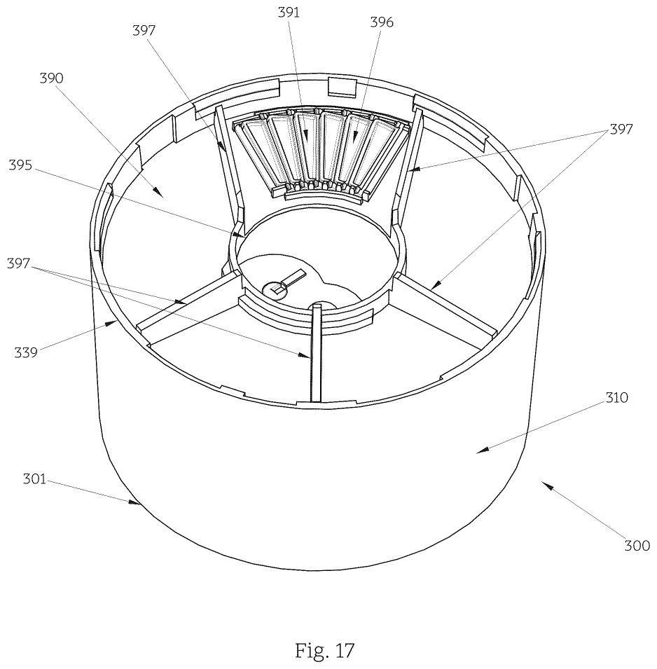

Turning to FIGS. 11, 12, 13, 14, 15-16, and 17, the cartridge 300 lower housing 310 includes frustoconical outer walls 311, inner walls 312, and a flat bottom wall 313 that form a tray basket. A stack of circular trays 370, such as comprising four trays 371, 372, 373, 374 are rotationally disposed in the tray basket to rotate about the axis of the inner walls 312 and outer walls 311. Steps 318 in the outer wall 311 rotationally support each tray by its tray flange 377 such that the trays are separated by a small gap. Each of the trays 371, 372, 373, 374 extends between an outer diameter wall 381 and an inner diameter wall 382 that are radially spaced apart from each other and a bottom wall 385 to a top edge 389. It should be appreciated that while embodiments herein illustrate a device 50 having four trays 371, 372, 373, 374, this is for example purposes and the claims should not be so limited. In other embodiments, the device 50 may have more or fewer trays.

Each of the circular trays in the tray stack 370 is subdivided into a number of bins 380, 387, such as 15 bins for example. A first segment is defined by a first number of loadable bins 380, alternatively referred to as fragrance-loaded bins. A second segment is defined by a second number of unloadable bins 387, alternatively referred to as non-loadable bins. Loadable bins 380 and unloadable bins 387 are defined by circumferential walls 383, 384 having a circumferential span 386. The circumferential walls 383, 384 extend from the tray base 385 to the tray top edge 389. The circumferential span of the bins may be a multiple or divisor of the circumferential span of the off-center opening 316 of the cartridge. The circumferential span of a number of loadable bins 380 forms a first segment. The circumferential span of a number of non-loadable bins 387 forms a second segment. The bottom of each bin 380, 397 has an array of airflow passages in the bottom wall 385, that is the bottom wall 385 may be a screen or sieve-like structure. The airflow passages in the bottom wall 385 of each loadable bin 380 are configured to support fragrance members, such as fragrance beads, and not allow fragrance members to fall out of the bin 380 while allowing airflow to pass around the fragrance member. In an embodiment, the fragrance members may be the same as those described in commonly owned PCT Application PCT/US2022/019289 entitled FRAGRANCE COMPOSITIONS, METHODS OF MANUFACTURE THEREOF AND ARTICLES COMPRISING THE SAME, the contents of which is incorporated by reference herein. A first number of loadable bins 380 in each tray are thus configured to receive a number of fragrance members A second number of bins 387 in each tray has walls 376 that extended from the bottom airflow passages 385 to the top edge 389 of the tray spaced closely enough to prevent loading of fragrance members while still allowing airflow to pass through the non-loadable bin 387. It should be noted that when rotated to certain positions, the stack of trays 370 form a closed airflow passage or duct from the off-center opening 316 through a number of loadable and unloadable bins 380, 387 up to the top of the tray stack such that the airflow will only pass through the bins 380, 387 directly above the off-center opening 316 and will not pass through other bins 380, 387 not directly above the off-center opening 316. The other bins 380, 387 not directly above the off-center opening are therefore effectively sealed off from fluidic communication with the airflow or surroundings.

The center opening 315 of the cartridge extends by inner walls 312 up to the cartridge outlet 317. The offset opening 316 extends by tray bin walls 381, 382, 383, 384 having a substantially same size, shape, and radial/circumferential location as the off-center opening 316 to the top of the tray stack 370. A tray stack cap 390 supported by the cartridge lower housing 310 forms a top wall of the top tray in the tray stack. The tray stack cap 390 has a center opening 395 and an off-center opening 396 having substantially same size, shape, and radial and circumferential locations as the center 315 and off-center 316 openings of the cartridge lower housing 310. The off-center opening 396 is covered by cartridge louvers 391 that are pivotably coupled in the tray stack cap 390. The cartridge louvers 391 are configured to pivot between a closed state in which the offset opening 396 of the tray stack cap 390 is blocked and an open state in which the offset opening 396 of the tray cap 390 is exposed. The cartridge louvers 391 are air biased to transition from the closed state to the open state. The cartridge louvers 391 are otherwise in the closed state. An upper cover 392, alternatively referred to as a top plate, that has a center opening 393 that is the same size as the cartridge outlet 317 is placed axially above the tray stack cap 390 and forces the air emitting from the two openings 395, 396 to be combined after flowing through the tray stack 370 before exiting the cartridge outlet 317. The tray stack cap 390 includes support ribs 394 that support the top plate 392.

A stack of axially spaced idler gears 350, preferably four, that have external gear teeth rotate on an axle 351, are positioned to engage the internal gear teeth 375 of each tray in the stack 370. The internal gear teeth 375 extend from the inner wall 382 in a circumferential array that is interrupted at a start 378 and end 379 positions. At the start position 378, the tray gear teeth 375 are blocked to prevent engagement with the idler gear 350 in advance of the start position. At the end position 379, the tray gear teeth 375 are removed so that after disengaging the last inner tooth 375, the idler gear turning will not continue to rotate the tray.

The tray drive shaft 340 is rotationally disposed within the cartridge 300 in a position to engage the stack of idler gears 350. The tray drive shaft 340 extends axially through the tray stack 370. The tray drive shaft 340 and idler gears 350 are radially smaller than the inner diameter wall 312 of the cartridge 300. The tray drive shaft 340 is configured to sequentially rotate each of the idler gears 350, which in turn rotates each tray in the tray stack 370 sequentially, described in greater detail below. The driveshaft 340 comprises a stack of, such as four for example, axially spaced interrupted gears 342. Each interrupted gear has a segment of gear teeth 343 with a first circumferential span and a locking ring 344 with a second circumferential span. In an embodiment, the second circumferential span of the locking ring is greater than 270 degrees. The gear teeth 343 of each of the interrupted gears 342 are oriented at intervals, such as 90 degrees for example, around the driveshaft 340 axis. This configuration sequentially moves each tray in the tray stack by one bin 380 for each partial turn, such as 90 degrees for example, of the driveshaft 340 as follows:

When initially assembled, one tray, such as the bottom tray 371 for example, of the tray stack 370 has a number, such as two for example, of loadable bins 380 located above and circumferentially aligned with the off-center cartridge opening 316. The other trays, such as 372, 373, 374 for example, have their unloadable bins 387 located above and circumferentially aligned with the off-center opening 316. Thus, before any rotation there are a number, such as two for example, bins 380 filled with fragrance members and a number, such as six for example, unfilled bins 387 receiving the airflow emitting from the off-center opening 316.

After a time interval determined by the system processor 510, user settings, and the cartridge memory 360, the system processor will command the motor 629 to rotate. This rotates the gear train 630. The output shaft 641 of the gear train 630 is coupled by tapered spline 642 to the mating tapered spline 341 of the cartridge driveshaft 340. The cartridge driveshaft 340, thus, rotates the same amount as the driveshaft gear 640. When the home switch 682 engages the timing mark 643 on the driveshaft gear 640, the motor 629 stops rotation. The gear train driveshaft gear 640 rotates the cartridge driveshaft 340 less than one turn, such as a quarter turn for example, before stopping. When the driveshaft 340 rotates less than one turn, only one set of teeth 343 on one of the driveshaft interrupted gears 342 will engage the teeth on one of the idler gears 350 and rotate the idler by a fixed amount determined by the number of teeth 343 on the interrupted gear 342 and the number of teeth on the idler gear 350. The idler gear 350 which is rotated will, in turn, rotate one of the trays in the tray stack 370 by an amount, such as corresponding to the angle of the bin 380 for example. The other trays remain stationary during this rotation because the teeth of the other idler gears 350 are continually engaged with the lockrings 344 of the interrupted gears 342 on the cartridge driveshaft 340.

The diameter of the lockrings 344 are set such that the flanks of two teeth of the idler gears 350 on either side of the plane defined between the between the driveshaft 340 axis and the idler axle 351 are nearly touching the lockring. Thus, the idler gears 350 cannot rotate freely because rotation would cause the idler gear teeth to intersect the lock ring 344; that is, the idler gears that are engaged by the lockrings 344 and not by the interrupted gear teeth 343 stay rotationally locked in position which, in turn, rotationally locks the trays that are not being turned by the interrupted gear teeth 343. After the first partial turn of the driveshaft, a fragrance loaded bin 380 of, for example, the second tray 372 rotates into, and a non-loadable bin 387 rotates out of, position above the off-center opening 316. After a second partial turn of the driveshaft 340, a fragrance loaded bin 380 of, for example, the third tray 373 rotates into, and a non-loadable bin 387 rotates out of, position above the off-center opening 316. After a third partial turn of the driveshaft 340, a fragrance loaded bin 380 of, for example, the fourth tray 374 rotates into and a non-loadable bin 387 rotates out of, position above the off-center opening 316. After a fourth turn of the driveshaft 340, a fragrance loaded bin 380 of, for example, the first tray 371 rotates into position above the off-center opening 316 and a fragrance loaded bin 380 of the first tray 371 that had previously been exposed to the airflow emitting from off-center opening 316 rotates out of position above the off-center opening 316 and no longer emits fragrance.

As partial turns of the driveshaft 340 continue, fragrance loaded bins 380 that have not been exposed to the airflow continue to replace fragrance loaded bins 380 and unloadable bins 387 that had previously been exposed to the airflow. At any one time, there may be a number, such as eight for example, fragrance loaded bins 380 positioned above the off-center opening that are providing fragrance into the airflow. The fragrance loaded bins 380 exposed to the airflow will vary from an oldest bin that will be replaced by the next partial turn of the driveshaft 340 to the newest bin that was the most recently moved into position over the off-center opening 316 with the last partial turn of the driveshaft 340. When the final unexposed fragrance loaded bin 380 is rotated into position above the off-center opening 316, the trays will be rotationally positioned such that the idler gears are aligned with the end of the tray internal teeth 379; thus, any continued rotation of the driveshaft 340 and idler gears 350 does not create additional rotation of any of the trays in the tray stack 370 because the idler gear teeth are no longer engaged with the tray teeth 375. Moreover, reverse rotation of the driveshaft does not reverse the rotation of the trays 371, 372, 373, 374, in the tray stack 370.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

Those of skill in the art will appreciate that various example embodiments are shown and described herein, each having certain features in the particular embodiments, but the present disclosure is not thus limited. Rather, the present disclosure can be modified to incorporate any number of variations, alterations, substitutions, combinations, sub-combinations, or equivalent arrangements not heretofore described, but which are commensurate with the scope of the present disclosure. Additionally, while various embodiments of the present disclosure have been described, it is to be understood that aspects of the present disclosure may include only some of the described embodiments. Accordingly, the present disclosure is not to be seen as limited by the foregoing description, but is only limited by the scope of the appended claims.

We claim:

1. A fragrance dispenser, comprising:
a housing extending from a bottom of the dispenser to a top of the dispenser, defining an air inlet at the bottom and including a cartridge basket at the top;
a cartridge removably disposed within the cartridge basket, the cartridge including a cartridge housing extending from a bottom to a top and defining an air inlet at the bottom and an outlet at the top, the cartridge including a tray stack of circular trays configured the same as each other, each of the circular trays having at least one bin configured to receive a fragrance member;
a tray drive shaft, within the cartridge, configured to sequentially rotate each of the trays about a tray stack center;
a drive unit, operationally coupled to the tray drive shaft; and
an air intake unit configured to draw air into the air inlet of the dispenser, into the air inlet of the cartridge, through the cartridge, and out of the air outlet of the cartridge.

2. The dispenser of claim 1, wherein:
the air inlet in the cartridge housing includes a center opening and an annular- or ring-section shaped opening radially offset and spaced apart from the center opening with a first circumferential span; and
each of the trays includes a center opening and an annular- or ring-section shaped first segment with first airflow passages, the first segment forming at least one bin, the bin having a second circumferential span.

3. The dispenser of claim 2, wherein:
the tray drive shaft, is configured to sequentially rotate each of the trays about the tray stack center by the circumferential span of one bin such that, in an initial rotational state and a final rotational state, the bins of each of the trays are circumferentially aligned with each other and with the offset opening of the cartridge housing.

4. The dispenser of claim 1, wherein:
each of the trays extends from a tray bottom to a tray top by a tray height;
the walls of the bins in the first segment each extend from a tray bottom to a tray top; and
the floor of the bins has openings to allow airflow but that will not allow fragrance members to pass through and has a height less than the tray height, the difference between the tray height and the floor height defining a bin depth.

5. The dispenser of claim 1, wherein:
bins in a second segment of a tray are configured to prevent the placement of the fragrance members therein.

6. The dispenser of claim 1, wherein:
each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and
the tray drive shaft extends axially through the tray stack, within the shaft opening, and is radially smaller than the inner diameter wall, so that a rotation axis of the tray drive shaft is offset from the tray stack center, the tray drive shaft having teeth that are axially spaced apart from each other for engaging the inward facing gear teeth of each of the respective ones of the trays.

7. The dispenser of claim 1, wherein:
each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and
the inward facing gear teeth of each of the trays engages an independently rotating idler gear, the axially spaced stack of idler gears extending axially through the tray stack, within the shaft opening, the idler gears being radially smaller than the inner diameter wall, so that a rotation axis of the idler gears is offset from the tray stack center; and
the tray driveshaft having an axial stack of interrupted gears engaging each idler gear, the tray drive shaft extends axially through the tray stack, within the shaft opening, and is radially smaller than the inner diameter wall, so that a rotation axis of the tray drive shaft is offset from the tray stack center.

8. The dispenser of claim 1, wherein:
each of the axially spaced interrupted gears on the driveshaft has a set of gear teeth of at least one tooth, spanning a circumferential segment less than 360 degrees for engaging the respective idler gear teeth, if present, or engaging the respective tray teeth for rotating the idler or tray when the teeth are engaged.

9. The dispenser of claim 1, wherein:
each of the axially spaced interrupted gears on the driveshaft has an extended ring or circular plate spanning a circumferential segment less than 360 degrees for engaging the teeth or semicircular grooves on the idler gears, if present, or engaging the teeth or semi-circular grooves in the tray to prevent the idler or tray from rotating while the ring or plate is engaged.

10. The dispenser of claim 1, wherein:
each set of teeth on the tray drive shaft interrupted gears are angularly offset from the teeth that are adjacent to it on the tray drive shaft such that the teeth of each interrupted gear engages the teeth of the respective idler gears or trays in sequence.

11. The dispenser of claim 1, wherein:
each of the trays in the tray stack extends between an outer diameter wall and an inner diameter wall that are radially spaced apart from each other, the inner diameter wall defining a shaft opening through the tray stack, and inward facing gear teeth are defined along inner diameter wall; and
the inward facing gear teeth on the trays have a blocked segment with a first circumferential span and a cut away segment with a second circumferential span, the first segment being placed to prevent the driveshaft or idler gear teeth from engaging the tray before the degree of initial rotation of the tray within the cartridge, the second segment being placed so that at the final degree of rotation; i.e. when the tray has rotated such that the last unused bin is positioned above the offset opening in the cartridge, the idler gear or driveshaft gear is positioned within the cutout and the teeth of the tray are disengaged from the teeth of the idler or driveshaft.

12. The dispenser of claim 1, wherein:
the dispenser includes an energy storage device and a reduction gear powered by the energy storage device, wherein the reduction gear includes an output gear, and
the tray drive shaft includes a bottom end that is formed with spline teeth and configured for engagement with the output spline of the reduction gear.

13. The dispenser of claim 1, wherein:
a tray cap of the cartridge is disposed above the top tray, the tray cap having a same size as the trays, the tray cap defines an offset opening that is aligned with the offset opening of the cartridge housing; and
cartridge louvers are pivotally coupled to the baseplate at the offset opening of the tray cap and configured to pivot between a closed state in which the offset opening of the tray cap is blocked and an open state in which the offset opening of the tray cap is exposed, wherein the cartridge louvers are air biased to transition from the closed state to the open state, and are otherwise in the closed state.

14. The dispenser of claim 1, wherein:
a top plate of the cartridge is disposed above the tray cap, the top plate having a center opening the same size as the center opening in the top cap, and the top cap having a center opening the same size as the center opening in the tray stack and an offset opening that is aligned with the offset opening of the cartridge housing, the combination of tray cap and top plate thus configured to guide air flowing through the offset opening and through the first or second segments of the tray stack to combine with air flowing through the center opening of the tray stack before flowing out of the cartridge.

15. The dispenser of claim 1, wherein:
the cartridge includes memory that stores flash memory data indicative of one or more of a type of fragrance member stored within the cartridge, fragrance member usage history, target speeds of the air movers, or a target speed of the tray drive shaft; and
the dispenser includes a processor that is configured to communicate with the memory on the cartridge and control a speed of the air movers, or the output gear responsive to the flash memory data.

16. The dispenser of claim 1, wherein:
the dispenser includes a fan configured to draw air into the inlet, and a motion sensor, and the processor in the dispenser is configured to activate the fan upon the motion sensor detecting motion.

17. The dispenser of claim 1, wherein:
the dispenser includes a fan configured to draw air into the inlet, and a motion sensor, and the processor in the dispenser is configured to predictively activate the fan based at least in part upon the timing history of signals from the motion sensor detecting motion or a preset schedule from a connected software application or processor firmware.

18. The dispenser of claim 17, wherein the predictive activation of the fan is based on machine learning.

19. The dispenser of claim 1, wherein:
the dispenser includes an LED array at the top of the housing and an LED array controller that is operationally connected to the processor in the housing and configured to illuminate upon settings based on at least one of, settings from capacitively sensed gestures on the device, or the motion sensor detecting motion.

20. The dispenser of claim 1, wherein:
the dispenser includes an LED array configured to illuminate the top of the cartridge.

21. The dispenser of claim 1, wherein:
the dispenser includes a ring-shaped lip under which a capacitive touch sensing circuit board is used to detect gestures by the user on the ring to adjust the operational settings for the dispenser.

22. The dispenser of claim 1, wherein:
the dispenser includes a baseplate disposed below the cartridge basket, the baseplate extends between an outer diameter edge and an inner diameter edge that are radially spaced apart from each other, the inner diameter edge defines a fan opening; and
a center fan is disposed with the fan opening, operationally connected to the processor in the dispenser, and configured to direct air through the shaft opening of the tray stack.

23. The dispenser of claim 1, wherein:
the baseplate of the dispenser defines an offset opening that is aligned with and with the offset opening of the cartridge housing, and a second fan is disposed below the offset opening, operationally connected to the processor in the dispenser, and configured to direct air through the offset opening.

24. The dispenser of claim 1, wherein:
housing louvers are pivotally coupled to the cartridge basket at the offset opening in the cartridge basket and configured to pivot between a closed state in which the offset opening is blocked and an open state in which the offset opening is exposed, wherein the housing louvers are air biased by the second fan to transition from the closed state to the open state, and are otherwise in the closed state.

25. The dispenser of claim 2, wherein:
housing louvers are pivotally coupled to the cartridge basket at the offset opening in the cartridge basket and configured to pivot between a closed state in which the offset opening is blocked and an open state in which the offset opening is exposed, wherein the housing louvers are air biased by the second fan to transition from the closed state to the open state, and are otherwise in the closed state.
